(19) United States
(12) Patent Application Publication
(10) Pub. No.: US 2017/0029398 A1
(43) Pub. Date: Feb. 2, 2017

(54) PROCESS FOR THE PREPARATION OF (1S)-1,5-ANHYDRO-1-C-{4-CHLORO-3-4[(4-ETHOXYPHENYL)METHYL]PHENYL]-GLUCITOL AND ITS SOLVATE THEREOF

(71) Applicants: Srinivasan THIRUMALAI RAJAN, Telangana, Hyderabad (IN); Sajja ESWARAIAH, Telangana, Hyderabad (IN); MSN LABORATORIES PRIVATE LIMITED, Telangana, Hyderabad (IN)

(72) Inventors: Srinivasan Thirumalai Rajan, Telangana, Hyderabad (IN); Sajja Eswaraiah, Telangana, Hyderabad (IN)

(21) Appl. No.: 15/303,577

(22) PCT Filed: Mar. 6, 2015
(86) PCT No.: PCT/IN2015/000119

§ 371 Date: Oct. 12, 2016

(30) Foreign Application Priority Data
Mar. 6, 2014 (IN) 1141/CHE/2014

(51) Int. Cl. C07D 309/10

(52) U.S. Cl. CPC C07D 309/10

(57) ABSTRACT

The present invention relates to a process for the preparation of (1S)-1,5-anhydro-1-C-[4-chloro-3-[(4-ethoxyphenyl)methyl]phenyl]-D-glucitol which is represented by the following structural formula-1 and its glycerol solvate.

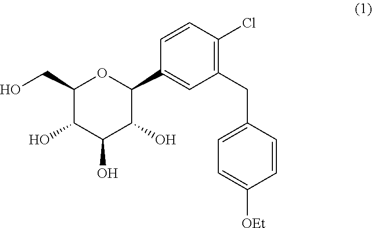 [SMILES: CCOc1ccc(Cc2cc([C@@H]3O[C@H](CO)[C@@H](O)[C@H](O)[C@H]3O)ccc2Cl)cc1]

### RELATED APPLICATIONS

This application claims the benefit of priority of our Indian patent application number 1141/CHE/2014 filed on 6 Mar. 2014 which is incorporated herein by reference.

### FIELD OF THE INVENTION

The present invention provides a process for the preparation of (1S)-1,5-anhydro-1-C-[4-chloro-3-[(4-ethoxyphenyl)methyl]phenyl]-D-glucitol which is represented by the following structural formula-1 and its glycerol solvate.

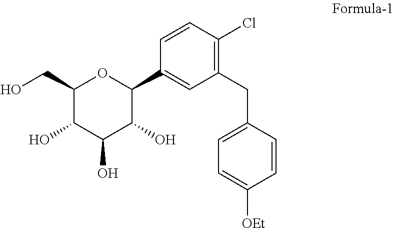 [SMILES: CCOc1ccc(Cc2cc([C@@H]3O[C@H](CO)[C@@H](O)[C@H](O)[C@H]3O)ccc2Cl)cc1]

### BACKGROUND OF THE INVENTION

(1S)-1,5-anhydro-1-C-[4-chloro-3-[(4-ethoxyphenyl)methyl]phenyl]-D-glucitol, commonly known as Dapagliflozin. Dapagliflozin is an inhibitor of sodium dependent glucose transporters, used to treat type 2 diabetes. It is developed by Bristol-Myers Squibb in partnership with AstraZeneca. Dapagliflozin was approved as (2S)-1,2-propane-diol monohydrate in United States on Jan. 8, 2014 and in Europe on Nov. 12, 2012.

Dapagliflozin and its process for the preparation were first disclosed in U.S. Pat. No. 6,515,117 (hereinafter referred as '117). One major step that is involved in the synthesis of Dapagliflozin is the purification of Dapagliflozin. The purification is done by converting the Dapagliflozin into tetra acetylated Dapagliflozin, which readily crystallizes. This compound upon treatment with LiOH.H₂O provides Dapaglilfozin as an amorphous glassy off-white solid with purity 94%.

Hence, there is a need in the art to develop Dapagliflozin with high pure and enhanced yield.

The problem is solved by the present invention by utilizing mild base such as sodium carbonate for deacetylation along with the formation of glycerol solvate of Dapaglilflozin.

### BRIEF DESCRIPTION OF THE INVENTION

The first aspect of the present invention is to provide (1S)-1,5-anhydro-1-C-[4-chloro-3-[(4-ethoxyphenyl)methyl]phenyl]-D-glucitol glycerol solvate.

The second aspect of the present invention is to provide a crystalline (1S)-1,5-anhydro-1-C-[4-chloro-3-[(4-ethoxyphenyl)methyl]phenyl]-D-glucitol glycerol solvate, hereinafter referred as crystalline form-M.

The third aspect of the present invention is to provide a process for the preparation of crystalline form-M of (1S)-1,5-anhydro-1-C-[4-chloro-3-[(4-ethoxyphenyl)methyl]phenyl]-D-glucitol glycerol solvate.

The fourth aspect of the present invention is to provide a process for the preparation of (1S)-1,5-anhydro-1-C-[4-chloro-3-[(4-ethoxyphenyl)methyl]phenyl]-D-glucitol compound of formula-1.

The fifth aspect of the present invention is to provide a process for the preparation of (1S)-1,5-anhydro-1-C-[4-chloro-3-[(4-ethoxyphenyl)methyl]phenyl]-D-glucitol compound of formula-1, comprising of treating (2R,3R,4R,5S,6S)-2-(acetoxymethyl)-6-(4-chloro-3-(4-ethoxybenzyl)phenyl) tetrahydro-2H-pyran-3,4,5-triyl triacetate compound of formula-7 with a mild base selected from alkali metal carbonates and bicarbonates in a suitable solvent to provide compound of formula-1.

The sixth aspect of the present invention is to provide a process for the preparation of (1S)-1,5-anhydro-1-C-[4-chloro-3-[(4-ethoxyphenyl)methyl]phenyl]-D-glucitol compound of formula-1 which proceeds through the glycerol solvate.

### BRIEF DESCRIPTION OF THE DRAWINGS

FIG. 1: Illustrates the powder X-ray diffraction pattern of crystalline form-M of (1S)-1,5-anhydro-1-C-[4-chloro-3-[(4-ethoxyphenyl)methyl]phenyl]-D-glucitol glycerol solvate.

FIG. 2: Illustrates the powder X-ray diffraction pattern of amorphous (1S)-1,5-anhydro-1-C-[4-chloro-3-[(4-ethoxyphenyl)methyl]phenyl]-D-glucitol.

### DETAILED DESCRIPTION OF THE INVENTION

The term “suitable solvent” used in the present invention is selected from, but not limited to “ester solvents” such as ethyl acetate, methyl acetate, isopropyl acetate, n-butyl acetate and the like; “ether solvents” such as tetrahydrofuran, dimethyl ether, diethyl ether, diisopropyl ether, methyl tert-butyl ether (MTBE), 1,4-dioxane and the like; “hydrocarbon solvents” such as toluene, hexane, heptane, pet ether, xylene, cyclohexane and the like; “polar aprotic solvents” such as dimethyl acetamide, dimethylsulfoxide, dimethylformamide, N-methyl-2-pyrrolidone and the like; “ketone solvents” such as acetone, methylethyl ketone, methylisobutyl ketone and the like; “alcoholic solvents” such as methanol, ethanol, n-propanol, isopropanol, n-butanol, isobutanol, tert-butanol and the like; “chloro solvents” such as dichloromethane, chloroform, dichloroethane, carbon tetrachloride and the like; “nitrile solvents” such as acetonitrile, butyronitrile, isobutyronitrile and the like; “polar solvent” such as water or mixtures thereof.

The term “solvate” used herein the present invention refers to a crystalline compound in which molecules of solvents are incorporated into the crystal lattice of Dapagliflozin. The term “glycerol solvate” refers to a crystalline dapagliflozin containing glycerol molecules in its crystal lattice.

The term “suitable base” used herein the present invention until unless specified is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; “alkali metal alkoxides” such as sodium methoxide, sodium ethoxide, sodium tert-butoxide, potassium methoxide, potassium ethoxide, potassium tert-butoxide and the like; ammonia; and organic bases such as triethyl amine, methyl amine, ethyl amine, 1,8-Diazabicyclo[5.4.0]undec-7-ene (DBU), 1,5-Diazabicyclo(4.3.0)non-5-ene (DBN), lithium dioisoporpylamide (LDA), n-butyl lithium, tribenzylamine, isopropyl amine, diisopropylamine (DIPA), diisopropylethylamine (DIPEA), N-methylmorpholine (NMP), N-ethylmorpholine, piperidine, dimethylaminopyridine (DMAP), morpholine, pyridine, 2,6-lutidine, 2,4,6-collidine, imidazole, 1-methylimidazole, 1,2,4-triazole, 1,4-diazabicyclo[2.2.2]octane (DABCO) or mixtures thereof.

The first aspect of the present invention provides (1S)-1,5-anhydro-1-C-[4-chloro-3-[(4-ethoxy phenyl)methyl]phenyl]-D-glucitol glycerol solvate.

The second aspect of the present invention provides a crystalline (1S)-1,5-anhydro-1-C-[4-chloro-3-[(4-ethoxyphenyl)methyl]phenyl]-D-glucitol glycerol solvate, hereinafter referred as crystalline form-M. The crystalline form-M is characterized by powder X-ray diffraction pattern having peaks at 4.1, 16.2, 20.3, 20.6 and 24.8±0.2 degrees of 2-theta.

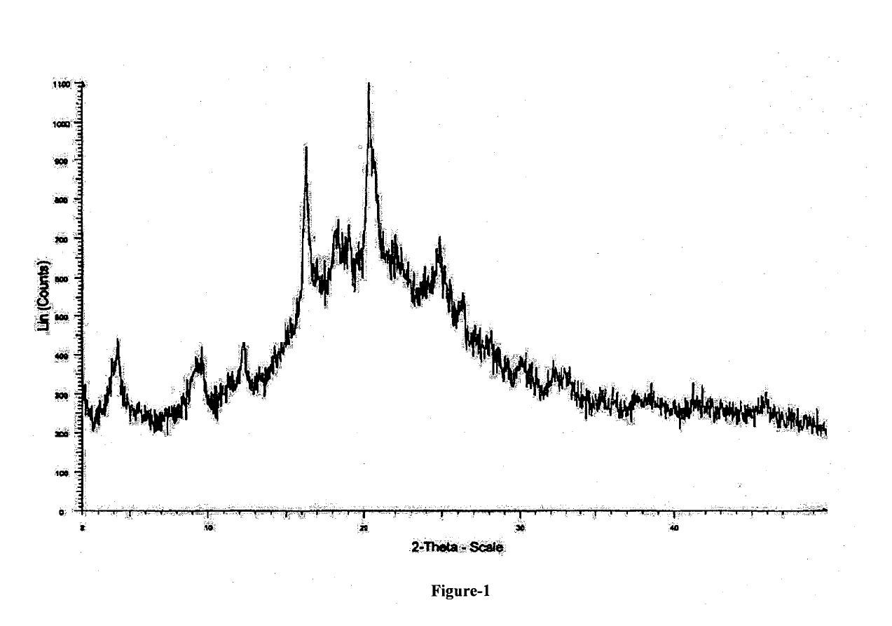

The said crystalline form-M is further characterized by its PXRD pattern as illustrated in FIG. 1.

The third aspect of the present invention provides a process for the preparation of crystalline form-M of (1S)-1,5-anhydro-1-C-[4-chloro-3-[(4-ethoxyphenyl)methyl]phenyl]-D-glucitol glycerol solvate, comprising of treating the (1S)-1,5-anhydro-1-C-[4-chloro-3-[(4-ethoxyphenyl)methyl]phenyl]-D-glucitol compound of formula-1 with glycerol in a suitable solvent selected from ether solvents, ester solvents, hydrocarbon solvents, alcoholic solvents, chloro solvents, ketone solvents, nitrile solvents, polar aprotic solvents, polar solvents or mixtures thereof.

A preferred embodiment of the present invention provides a process for the preparation of crystalline form-M of (1S)-1,5-anhydro-1-C-[4-chloro-3-[(4-ethoxyphenyl)methyl]phenyl]-D-glucitol glycerol solvate, comprising of treating the (1S)-1,5-anhydro-1-C-[4-chloro-3-[(4-ethoxyphenyl)methyl]phenyl]-D-glucitol compound of formula-1 with glycerol in water.

The fourth aspect of the present invention provides a process for the preparation of (1S)-1,5-anhydro-1-C-[4-chloro-3-[(4-ethoxyphenyl)methyl]phenyl]-D-glucitol compound of formula-1, comprising of:
a) Reacting the (3R,4S,5S,6R)-3,4,5-trihydroxy-6-(hydroxymethyl)tetrahydro-2H-pyran-2-one compound of formula-8 with trimethyl silyl chloride in presence of N-methyl morpholine in tetrahydrofuran to provide (3R,4S,5R,6R)-3,4,5-tris(trimethylsilyloxy)-6-((trimethylsilyloxy)methyl)tetrahydro-2H-pyran-2-one compound of formula-5,
b) reacting the compound of formula-5 in-situ with 4-bromo-1-chloro-2-(4-ethoxybenzyl) benzene compound of formula-4 in presence of n-butyl lithium in tetrahydrofuran, followed by treating the obtained compound with methane sulfonic acid in methanol to provide (2S,3R,4S,5S,6R)-2-(4-chloro-3-(4-ethoxybenzyl) phenyl)-6-(hydroxylmethyl)-2-methoxytetrahydro-2H-pyran-3,4,5-triol compound of formula-6,
c) reacting the compound of formula-6 with triethyl silane in presence of BF₃-etherate in a mixture of dichloromethane and acetonitrile to provide (1S)-1,5-anhydro-1-C-[4-chloro-3-[(4-ethoxy phenyl)methyl]phenyl]-D-glucitol compound of formula-1,
d) reacting the compound of formula-1 in-situ with acetic anhydride in presence of dimethylamino pyridine in dichloromethane, purifying the obtained compound using methanol to provide (2R,3R,4R,5S,6S)-2-(acetoxymethyl)-6-(4-chloro-3-(4-ethoxy benzyl)phenyl)tetrahydro-2H-pyran-3,4,5-triyl triacetate compound of formula-7,
e) treating the compound of formula-7 with a mild base selected from alkali metal carbonates and bicarbonates in a suitable solvent to provide compound of formula-1,
f) converting the compound of formula-1 into its glycerol solvate by treating it with glycerol in a suitable solvent,
g) dissolving the glycerol solvate in a suitable solvent, washing the reaction mixture with water and then distilling off the solvent to provide pure compound of formula-1.

Wherein, the suitable solvent used in step-e) & step-f) is selected from ether solvents, ester solvents, alcoholic solvents, chloro solvents, ketone solvents, hydrocarbon solvents, polar aprotic solvents, nitrile solvents, polar solvents (or) mixtures thereof; and in step-g) the solvent is same as defined in step-e) & f) excluding alcoholic solvents and polar solvents.

A preferred embodiment of the present invention provides a process for the preparation of (2S,3R,4R,5S,6R)-2-(4-chloro-3-(4-ethoxybenzyl)phenyl)-6-(hydroxymethyl) tetrahydro-2H-pyran-3,4,5-triol compound of formula-1, comprising of:
a) Reacting the (3R,4S,5S,6R)-3,4,5-trihydroxy-6-(hydroxymethyl)tetrahydro-2H-pyran-2-one compound of formula-8 with trimethylsilyl chloride in presence of N-methyl morpholine in tetrahydrofuran to provide (3R,4S,5R,6R)-3,4,5-tris(trimethyl silyloxy)-6-((trimethylsilyloxy)methyl)tetrahydro-2H-pyran-2-one compound of formula-5,
b) reacting the compound of formula-5 in-situ with 4-bromo-1-chloro-2-(4-ethoxybenzyl) benzene compound of formula-4 in presence of n-butyl lithium in tetrahydrofuran, followed by treating the obtained compound with methane sulfonic acid in methanol to provide (2S,3R,4S,5S,6R)-2-(4-chloro-3-(4-ethoxybenzyl) phenyl)-6-(hydroxymethyl)-2-rnethoxytetrahydro-2H-pyran-3,4,5-triol compound of formula-6,
c) reacting the compound of formula-6 with triethyl silane in presence of BF₃-etherate in a mixture of dichloromethane and acetonitrile to provide (1S)-1,5-anhydro-1-C-[4-chloro-3-[(4-ethoxyphenyl)methyl]phenyl]-D-glucitol compound of formula-1,
d) reacting the compound of formula-1 in-situ with acetic anhydride in presence of dimethylamino pyridine in dichloromethane, purifying the obtained compound using methanol to provide (2R,3R,4R,5S,6S)-2-(acetoxymethyl)-6-(4-chloro-3-(4-ethoxy benzyl)phenyl)tetrahydro-2H-pyran-3,4,5-triyl triacetate compound of formula-7,
e) treating the compound of formula-7 with sodium carbonate in aqueous methanol to provide compound of formula-1,
f) converting the compound of formula-1 into its glycerol solvate by treating it with glycerol in water,
g) dissolving the glycerol solvate compound of formula-1 in ethyl acetate, washing the reaction mixture with water and then distilling off the solvent to provide pure compound of formula-1.

In the above aspect, methyl tertiarybutyl ether (MTBE) is also used in step-g) to get the pure compound of formula-1.

The 4-bromo-1-chloro-2-(4-ethoxybenzyl)benzene compound of formula-4 used in the above aspect of the present invention can be prepared by the following steps of:
a) Converting the 5-bromo-2-chlorobenzoic acid compound of formula-2 into its acid chloride by treating it with thionyl chloride in a mixture of dichloromethane and dimethylformamide,
b) reacting the acid chloride in-situ with phenetol in presence of aluminium chloride in dichloromethane, purifying the obtained compound using methanol to provide (5-bromo-2-chlorophenyl)(4-ethoxyphenyl)methanone compound of formula-3,
c) reducing the compound of formula-3 with triethylsilane in presence of titanium tetrachloride in dichloromethane, purifying the obtained compound using methanol to provide 4-bromo-1-chloro-2-(4-ethoxybenzyl)benzene compound of formula-4.

US '117 patent disclosed a process for the preparation of 4-bromo-1-chloro-2-(4-ethoxybenzyl) benzene compound of formula-4, which involves the reduction of (5-bromo-2-chlorophenyl)(4-ethoxyphenyl)methanone compound of formula-3 using triethylsilane in presence of BF₃-etherate in a mixture of dichloromethane and acetonitrile and provides compound of formula-4 with yield of 60 to 65%. Whereas in the present invention, triethylsilane used in presence of titanium tetrachloride (in place of BF₃-etherate) which increases the yield of the compound of formula-4 to 85-90%. Hence the present invention is advantageous over the processes of the prior art.

The above aspects of the present invention are schematically represented as follows:

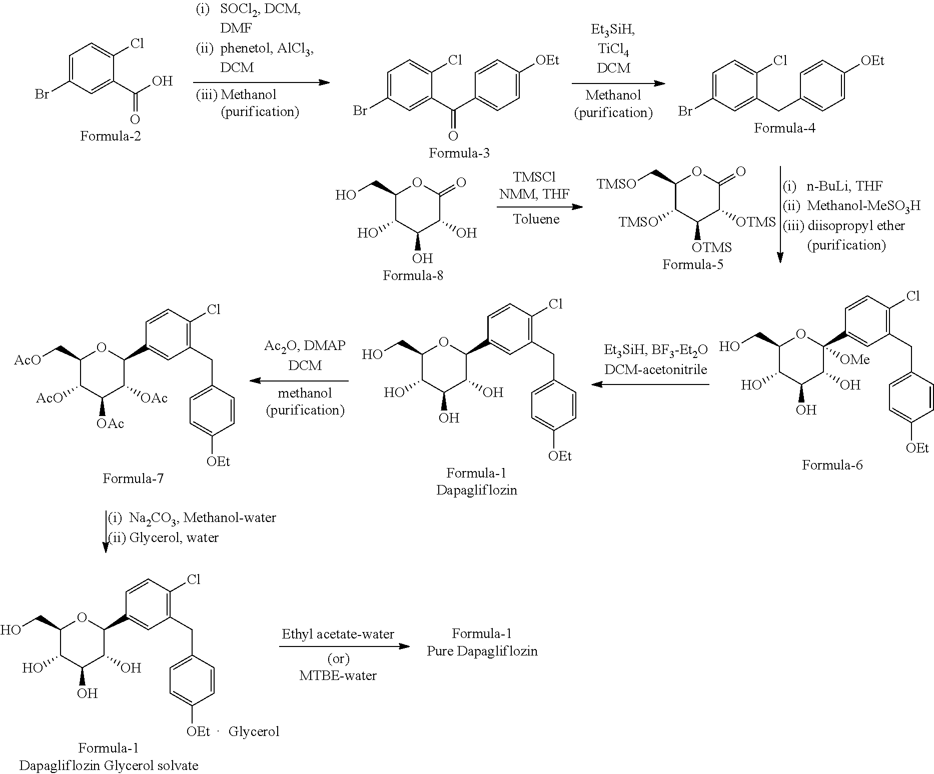

The 5-bromo-2-chlorobenzoic acid compound of formula-2 and (3R,4S,5S,6R)-3,4,5-trihydroxy-6-(hydroxymethyl)tetrahydro-2H-pyran-2-one compound of formula-8 are commercially available.

The fifth aspect of the present invention provides a process for the preparation of (1S)-1,5-anhydro-1-C-[4-chloro-3-[(4-ethoxyphenyl)methyl]phenyl]-D-glucitol compound of formula-1, comprising of treating (2R,3R,4R,5S,6S)-2-(acetoxymethyl)-6-(4-chloro-3-(4-ethoxy benzyl)phenyl)tetrahydro-2H-pyran-3,4,5-triyl triacetate compound of formula-7 with a mild base selected from alkali metal carbonates and bicarbonates in a suitable solvent to provide compound of formula-1.

Wherein, the suitable solvent is selected from hydrocarbon solvents, ether solvents, ester solvents, polar aprotic solvents, alcoholic solvents, ketone solvents, chloro solvents, nitrile solvents, polar solvents (or) mixtures thereof.

The preferred embodiment of the present invention provides a process for the preparation of (1S)-1,5-anhydro-1-C-[4-chloro-3-[(4-ethoxyphenyl)methyl]phenyl]-D- glucitol compound of formula-1, comprising of treating (2R,3R,4R,5S,6S)-2-(acetoxymethyl)-6-(4-chloro-3-(4-ethoxybenzyl)phenyl)tetrahydro-2H-pyran-3,4,5-triyl triacetate compound of formula-7 with sodium carbonate in aqueous methanol to provide compound of formula-1.

U.S. Pat. No. 7,919,598 disclosed a process for the preparation of dapagliflozin. This process involves the usage of sodium hydroxide as a base for deacetylation of compound of formula-7. When the same process was carried out in our laboratory, the compound of formula-1 is obtained with purity of 94.54% and yield: 73.6%. Use of strong bases might be leading to degradation. When the above process is carried out in our laboratory using mild bases such as alkali metal carbonates (or) bicarbonates, preferably sodium carbonate, we surprisingly found that the purity and yield of compound of formula-1 significantly increased to 99.08% by HPLC and 96.18% yield respectively. Hence the present invention is more advantageous.

The sixth aspect of the present invention provides a process for the preparation of pure amorphous (1S)-1,5-anhydro-1-C-[4-chloro-3-[(4-ethoxyphenyl)methyl]phenyl]-D-glucitol compound of formula-1, comprising of:
a) Treating (2R,3R,4R,5S,6S)-2-(acetoxymethyl)-6-(4-chloro-3-(4-ethoxybenzyl) phenyl)tetrahydro-2H-pyran-3,4,5-triyl triacetate compound of formula-7 with a mild base selected from alkali metal carbonates and bicarbonates in a suitable solvent to provide (1S)-1,5-anhydro-1-C-[4-chloro-3-[(4-ethoxyphenyl)methyl]phenyl]-D-glucitol compound of formula-1,
b) converting the compound of formula-1 into its glycerol solvate by treating it with glycerol in a suitable solvent,
c) dissolving the obtained glycerol solvate compound of formula-1 in a suitable solvent, washing the reaction mixture with water and then distilling off the solvent to provide pure amorphous compound of formula-1.

Wherein, the suitable solvent used in step-b) is selected from ether solvents, ester solvents, nitrile solvents, alcoholic solvents, polar aprotic solvents, polar solvents, ketone solvents, chloro solvents, hydrocarbon solvents or mixtures thereof; and in step-c) the solvent is same as defined in step-b) excluding alcoholic solvents and polar solvents.

The preferred embodiment of the present invention provides a process for the preparation of pure amorphous (2S,3R,4R,5S,6R)-2-(4-chloro-3-(4-ethoxybenzyl)phenyl)-6-(hydroxyl methyl)tetrahydro-2H-pyran-3,4,5-triol compound of formula-1, comprising of:
a) Treating (2R,3R,4R,5S,6S)-2-(acetoxymethyl)-6-(4-chloro-3-(4-ethoxybenzyl) phenyl)tetrahydro-2H-pyran-3,4,5-triyl triacetate compound of formula-7 with sodium carbonate in aqueous methanol to provide (1S)-1,5-anhydro-1-C-[4-chloro-3-[(4-ethoxyphenyl)methyl]phenyl]-D-glucitol compound of formula-1,
b) converting the compound of formula-1 into its glycerol solvate by treating it with glycerol in water,
c) dissolving the obtained glycerol solvate compound of formula-1 in ethyl acetate, washing the reaction mixture with water and then distilling off the solvent to provide pure amorphous compound of formula-1.

In another preferred embodiment of the present invention provides a process for the preparation of pure amorphous (2S,3R,4R,5S,6R)-2-(4-chloro-3-(4-ethoxybenzyl)phenyl)-6-(hydroxyl methyl)tetrahydro-2H-pyran-3,4,5-triol compound of formula-1, comprising of:
a) Treating (2R,3R,4R,5S,6S)-2-(acetoxymethyl)-6-(4-chloro-3-(4-ethoxybenzyl) phenyptetrahydro-2H-pyran-3,4,5-triyl triacetate compound of formula-7 with sodium carbonate in aqueous methanol to provide (1S)-1,5-anhydro-1-C-[4-chloro-3-[(4-ethoxyphenyl)methyl]phenyl]-D-glucitol compound of formula-1,
b) converting the compound of formula-1 into its glycerol solvate by treating it with glycerol in water,
c) dissolving the obtained glycerol solvate compound of formula-1 in methyl tertiarybutyl ether (MTBE), washing the reaction mixture with water and then distilling off the solvent to provide pure amorphous compound of formula-1.

The Dapagliflozin obtained by the known process is having purity about 99.08% by HPLC. Whereas, the Dapagliflozin of the present invention proceed through the glycerol solvate which enhances the purity by 99.6% by HPLC. Hence the present invention is advantageous over the processes disclosed in the art.

The above obtained highly pure (1S)-1,5-anhydro-1-C-[4-chloro-3-[(4-ethoxyphenyl) methyl]phenyl]-D-glucitol compound of formula-1 of the present invention can be utilized for the preparation of (1S)-1,5-anhydro-1-C-[4-chloro-3-[(4-ethoxyphenyl)methyl]phenyl]-D-glucitol as well as its (S)-1,2-propane-diol monohydrate.

The impurities which were observed during the synthesis of compound of formula-1 are represented by following structural formula.

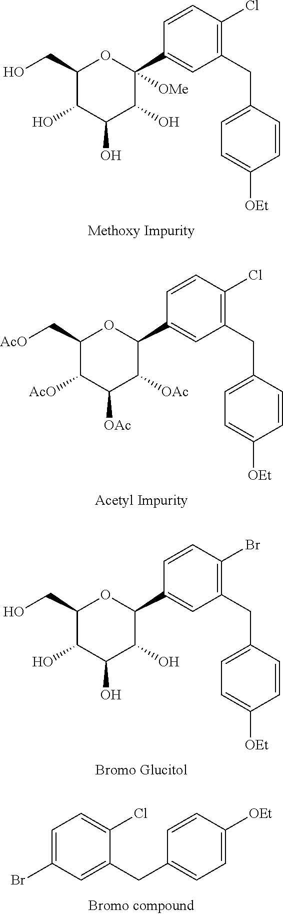 [SMILES: CCOc1ccc(Cc2cc([C@]3(OC)O[C@H](CO)[C@@H](O)[C@H](O)[C@H]3O)ccc2Cl)cc1 ; CCOc1ccc(Cc2cc([C@@H]3O[C@H](COC(C)=O)[C@@H](OC(C)=O)[C@H](OC(C)=O)[C@H]3OC(C)=O)ccc2Cl)cc1 ; CCOc1ccc(Cc2cc([C@@H]3O[C@H](CO)[C@@H](O)[C@H](O)[C@H]3O)ccc2Br)cc1 ; CCOc1ccc(Cc2cc(Br)ccc2Cl)cc1]

The bromo Glucitol impurity can be prepared by the following synthetic scheme.

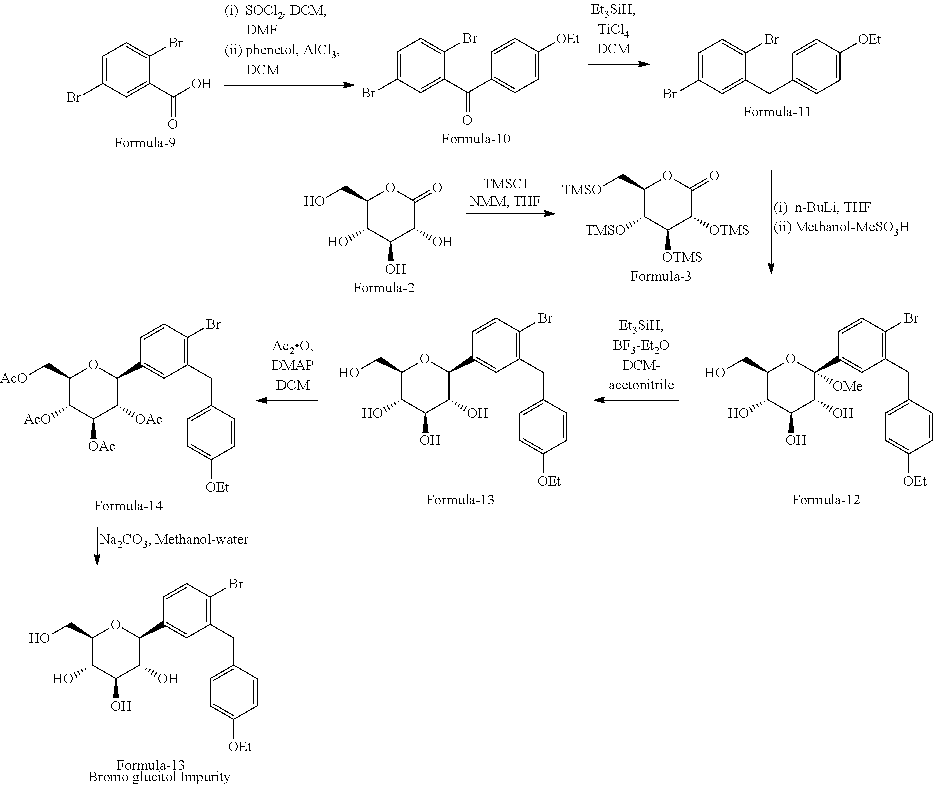

(1S)-1,5-anhydro-1-C-[4-chloro-3-[(4-ethoxyphenyl)methyl]phenyl]-D-glucitol compound of formula-1 obtained by the present invention is having purity about 99.6% by HPLC and controls all the impurities below ICH limits.

PXRD analysis of the crystalline (1S)-1,5-anhydro-1-C-[4-chloro-3-[(4- ethoxyphenyl)methyl]phenyl]-D-glucitol glycerol solvate and PXRD analysis of the compound of formula-1 of the present invention was carried out using BRUKER/AXS X-Ray diffractometer using Cu Kα radiation of wavelength 1.5406 A°.

(1S)-1,5-anhydro-1-C-[4-chloro-3-[(4-ethoxyphenyl)methyl]phenyl]-D-glucitol and its related substances are measured by using HPLC with the following chromatographic conditions:

Apparatus: A liquid chromatograph is equipped with variable wavelength UV-detector; Column: Durashell C18, 250×4.6 mm, 5 μm 100° A or equivalent; Flow rate: 1.2 ml/min; Elution: Gradient; Wavelength: 225 nm; Column temperature: 40° C.; Injection volume: 10 μL; Run time: 45 mins; Needle wash: Diluent; Diluent: Acetonitrile:water (90:10 v/v); Mobile phase A: Buffer (100%); Mobile phase B: Acetonitrile:Water (90:10 v/v); Buffer preparation: Transfer about 1.0 ml of ortho phosphoric acid (85%) into 1000 ml of mill-Q-water and mix well. Filter this solution through 0.22 μm nylon membrane filter paper.

Dapaglifiozin and its glycerol solvate obtained by the present invention can be further micronized or milled by the conventional methods to get the desired particle size to achieve desired solubility profile based on different forms of pharmaceutical composition requirements. Techniques that may be used for particle size reduction include, but not limited to ball, roller and hammer mills, and jet mills. Milling or micronization may be performed before drying, or after the completion of drying of the product.

The best mode of carrying out the present invention is illustrated by the below mentioned examples. These examples are provided as illustration only and hence should not be construed as limitation of the scope of the invention.

### EXAMPLES

### Example-1

### Preparation of (5-bromo-2-chlorophenyl)(4-ethoxyphenyl)methanone (Formula-3)

Thionyl chloride (194.78 ml) was slowly added to a mixture of 5-bromo-2-chlorobenzoic acid compound of formula-2 (200 gms), dichloromethane (1000 ml) and dimethylformamide (1 ml) at 25-30° C. Heated the reaction mixture to 35-40° C. and stirred for 4 hrs at the same temperature. Distilled off the solvent completely from the reaction mixture under reduced pressure. Dichloromethane (1600 ml) was added to the obtained compound at 25-30° C. and stirred for 20 mins at the same temperature. Cooled the reaction mixture to 5-10° C. and stirred for 15 mins at the same temperature. Aluminium chloride (110.9 gms) was slowly added to the reaction mixture at 5-10° C., the temperature of the reaction mixture was raised to 25-30° C. and stirred for 10 mins at the same temperature. Phenetole (103.5 gms) was slowly added to the reaction mixture at 25-30° C. and stirred for 10 hrs at the same temperature. The reaction mixture was poured into chilled hydrochloric acid solution (1000 ml of hydrochloric acid in 1000 gms of ice) at 25-30° C. and stirred for 20 mins at the same temperature. Separated the both organic and aqueous layers, the organic layer was washed with 5% aqueous sodium bicarbonate solution followed by with 10% aqueous sodium chloride solution. Distilled off the solvent completely from the organic layer under reduced pressure. Methanol (400 ml) was added to the obtained compound at 55-60° C. and stirred for 45 mins. Cooled the reaction mixture to 0-5° C. and stirred for 2 hrs at the same temperature. Filtered the precipitated solid, washed with methanol. Methanol (500 ml) was added to the wet solid, heated to 65-70° C. and stirred for 1 hr 30 mins at the same temperature. Cooled the reaction mixture to 25-30° C., then to 0-5° C. and stirred for 3 hrs at 0-5° C. Filtered the precipitated solid, washed with methanol and then dried to get title compound.

Yield: 178 gms; Melting range: 68-72° C.; Purity by HPLC: 98.8%.

### Example-2

### Preparation of 4-bromo-1-chloro-2-(4-ethoxybenzyl)benzene (Formula-4)

Titanium tetrachloride (193.8 ml) was added to a mixture of (5-bromo-2-chlorophenyl)(4-ethoxyphenyl)methanone compound of formula-3 (200 gms) and dichloromethane (2000 ml) under nitrogen atmosphere at 25-30° C. and stirred for 15 mins at the same temperature. Cooled the reaction mixture to 0-5° C. and triethylsilane (281.8 ml) was slowly added to it at 0-5° C. The temperature of the reaction mixture was raised to 25-30° C. and stirred for 8 hrs at the same temperature. Cooled the reaction mixture to 0-5° C. and chilled water was slowly added to the reaction mixture. Raised the temperature of the reaction mixture to 25-30° C. and stirred for 30 mins at 25-30° C. Separated the both organic and aqueous layers, the organic layer was washed with 10% aqueous sodium hydroxide solution followed by with 10% aqueous sodium chloride solution. Distilled off the solvent from the organic layer completely under reduced pressure at a temperature below 45° C. Toluene was added to the obtained compound, heated to 125-130° C. and stirred for 8 hrs at the same temperature under azeotropic condition. Cooled the reaction mixture to 60-65° C. and then distilled off the solvent completely under reduced pressure at a temperature below 70° C. Methanol (700 ml) was added to the obtained compound at 35-40° C. and stirred for 30 mins at the same temperature. Cooled the reaction mixture to 25-30° C. and stirred for 45 mins at the same temperature. The reaction mixture was further cooled to −5 to 0° C. and stirred for 2 hrs at the same temperature. Filtered the precipitated solid, washed with methanol and then dried to get title compound.

Yield: 165 gms; Melting range: 42-45° C.; purity by HPLC: 99.2%.

### Example-3

### Preparation of (2S,3R,4S,5S,6R)-2-(4-chloro-3-(4-ethoxybenzyl)phenyl)-6-(hydroxymethyl)-2-methoxytetrahydro-2H-pyran-3,4,5-triol (Formula-6)

Step-a) Preparation of (3R,4S,5R,6R)-3,4,5-tris(trimethylsilyloxy)-6-((trimethylsilyloxy) methyl)tetrahydro-2H-pyran-2-one (Formula-5)

N-methyl morpholine (560 ml) was added to a mixture of (3R,4S,5S,6R)-3,4,5-trihydroxy-6-(hydroxymethyl)tetrahydro-2H-pyran-2-one compound of formula-8 (100 gms) and tetrahydrofuran (1000 ml) at 25-30° C. Cooled the reaction mixture to 0-5° C. and stirred for 20 mins at the same temperature. Trimethylsilyl chloride (538 ml) was added slowly to the reaction mixture at 0-5° C., heated the reaction mixture to 45-50° C. and stirred for 12 hrs at the same temperature. Cooled the reaction mixture to −15 to −10° C. Toluene followed by chilled water were added to the reaction mixture at a temperature below −5° C. Raised the temperature of the reaction mixture to 25-30° C. and stirred for 15 mins at the same temperature. Separated both the organic and aqueous layers, washed the organic layer with 10% aqueous sodium dihydrogen phosphate dihydrate solution followed by 10% aqueous sodium chloride solution. Distilled off the solvent completely from the organic layer under reduced pressure. Kept the obtained compound at 0-5° C. and can be utilized for next step.

Step-b) Preparation of (2S,3R,4S,5S,6R)-2-(4-chloro-3-(4-ethoxybenzyl)phenyl)-6-(hydroxymethyl)-2-methoxytetrahydro-2H-pyran-3,4,5-triol (Formula-6)

A mixture of 4-bromo-1-chloro-2-(4-ethoxybenzyl)benzene compound of formula-4 (83.33 gms) and toluene (420 ml) was heated to reflux temperature and stirred for 2 hrs under azeotropic conditions. Distilled off the solvent completely under reduced pressure. Cooled the obtained compound to 25-30° C. under nitrogen atmosphere. Tetrahydrofuran (665 ml) followed by the compound obtained in step-(a) were added to the reaction mixture at 25-30° C. under nitrogen atmosphere. Cooled the reaction mixture to −85 to −80° C. and stirred for 20 mins at the same temperature. n-butyl lithium (238.3 ml) was slowly added to the reaction mixture at −85 to −80° C. under nitrogen atmosphere. Raised the temperature of the reaction mixture to −75 to −70° C. and stirred for 2 hrs at the same temperature. A solution of methane sulfonic acid (91.4 ml) in methanol (500 ml) was slowly added to the reaction mixture at −75 to −70° C. The temperature of the reaction mixture was slowly raised to 0-5° C. and then to 10-15° C. The reaction mixture was stirred for 18 hrs at 10-15° C. 10% aqueous sodium bicarbonate solution was added to the reaction mixture at 10-15° C. The temperature of the reaction mixture was raised to 25-30° C. and stirred for 15 mins. Separated the both organic and aqueous layers, the aqueous layer was extracted with ethyl acetate. Both the organic layers were combined, washed with 10% aqueous sodium chloride solution and then distilled off the solvent completely from the organic layer under reduced pressure. Cooled the obtained compound to 40-45° C. and then co-distilled with toluene. Toluene (100 ml) was added to the obtained compound at 25-30° C. and stirred for 20 mins at the same temperature. Diisopropyl ether (500 ml) was added to the reaction mixture at 25-30° C. Cooled the reaction mixture to 15-20° C. and stirred for 2 hrs at the same temperature. Settled the reaction mixture and decanted the upper organic layer. Distilled off the solvent from the bottom to get title compound. Yield: 135 gms; Purity by HPLC: 89.02%.

### Example-4

### Preparation of (2R,3R,4R,5S,6S)-2-(acetoxymethyl)-6-(4-chloro-3-(4-ethoxy benzyl)phenyl)tetrahydro-2H-pyran-3,4,5-triyl triacetate (Formula-7)

Step-a) Preparation of (1S)-1,5-anhydro-1-C-[4-chloro-3-[(4-ethoxyphenyl)methyl]phenyl]-D-glucitol (Formula-1)

Dichloromethane (1250 ml) followed by acetonitrile (1250 ml) were added to (2S ,3R,4 S,5 S ,6R)-2-(4-chloro-3-(4-ethoxybenzyl)phenyl)-6-(hydroxymethyl)-2-methoxy tetrahydro-2H-pyran-3,4,5-triol compound of formula-6 (250 gms) at 25-30° C. Cooled the reaction mixture to −20 to −25° C. under nitrogen atmosphere and stirred for 15 mins at the same temperature. Triethylsilane (132.35 gms) was slowly added to the reaction mixture at −20 to −25° C. and stirred for 15 mins. BF₃-etherate (193.5 gms) was added to the reaction mixture at −25 to −20° C. and stirred for 15 mins at the same temperature. The temperature of the reaction mixture was slowly raised to −5 to 0° C. and stirred for 1 hr at the same temperature. The pH of the reaction mixture was neutralized by using 10% aqueous sodium bicarbonate solution. Ethyl acetate was added to the reaction mixture and stirred for 15 mins. Separated the both organic and aqueous layers, washed the organic layer with aqueous sodium chloride solution (50 gms of sodium chloride in 1250 ml of water) and then distilled off the solvent completely from the organic layer under reduced pressure.

Step-b) Preparation of (2R,3R,4R,5S,6S)-2-(acetoxymethyl)-6-(4-chloro-3- (4-ethoxy benzyl)phenyl)tetrahydro-2H-pyran-3,4,5-triyl triacetate (Formula-7)

Dichloromethane (1000 ml) was added to the obtained compound in step-a) at 25-30° C. and stirred for 15 mins at the same temperature. Dimethylaminopyridine (11.94 gms) was added to the reaction mixture at 25-30° C. and stirred for 20 mins at the same temperature. Acetic anhydride (249.46 gms) was added to the reaction mixture at 25-30° C. and stirred for 4 hrs at the same temperature. Water was slowly added to the reaction mixture at 25-30° C. and stirred for 15 mins. Both the organic and aqueous layers were separated and the organic layer was washed with aqueous 10% aqueous hydrochloric acid solution, followed by with 10% aqueous sodium bicarbonate solution and then with 10% aqueous sodium chloride solution. Distilled off the solvent completely from the organic layer under reduced pressure. Methanol (1000 ml) was added to the obtained compound at 35-40° C., heated the reaction mixture to 60-65° C. and stirred for 2 hrs at the same temperature. Cooled the reaction mixture to 25-30° C. and stirred for 30 mins. The reaction mixture was further cooled to 0-5° C. and stirred for 1 hr 30 mins at the same temperature. Filtered the solid, washed with methanol. Methanol (1200 ml) was added to the obtained solid, heated to reflux temperature and stirred for 45 mins. Cooled the reaction mixture to 0-5° C. and stirred for 1 hr 30 mins. Filtered the solid, washed with methanol and then dried to get the title compound. Yield: 128 gms; MR: 125-128° C.; Purity by HPLC: 99.53%.

### Example-5

### Preparation of Glycerol Solvate of (1S)-1,5-anhydro-1-C-[4-chloro-3-[(4-ethoxyphenyl)methyl]phenyl]-D-glucitol

A mixture of (2R,3R,4R,5S ,6S)-2-(acetoxymethyl)-6-(4-chloro-3 - ethoxybenzyl) phenyltetrahydro-2H-pyran-3,4,5-triyl triacetate compound of formula-7 (100 gm), methanol (900 ml) and water (100 ml) was stirred for 30 minutes at 25-30° C. Sodium carbonate (165.3 gm) was added to the reaction mixture at 25-30° C., heated to 45-50° C. and stirred the reaction mixture for 10 hrs at the same temperature. Cooled the reaction mixture to 25-30° C. and stirred for 15 minutes at the same temperature. Filtered the reaction mixture, washed with methanol and then distilled off the solvent completely from the filtrate under reduced pressure. Water was added to the obtained compound at 25-30° C. and stirred for 20 minutes at the same temperature. Decanted water layer from the reaction mixture. Water (1000 ml) was added to the obtained compound at 25-30° C. and stirred for 20 minutes at the same temperature. Heated the reaction mixture to 80-85° C. and stirred for 1 hr 30 minutes. Cooled the reaction mixture to 25-30° C. and stirred for 20 minutes at the same temperature. Glycerol (19.15 gm) was added to the reaction mixture at 25-30° C. and stirred the reaction mixture for 30 minutes at the same temperature. Cooled the reaction mixture to 0-5° C. and stirred for 6 hrs at the same temperature. Filtered the precipitated solid and washed with water to get (1S)-1,5-anhydro-1-C-[4-chloro-3-[(4-ethoxyphenyl)methyl]phenyl]-D-glucitol glycerol solvate.

### Example-6

### Preparation of (1S)-1,5-anhydro-1-C-[4-chloro-3-[(4-ethoxyphenyl)methyl]phenyl]-D-glucitol Compound of formula-1 Through Glycerol Solvate Formation Using Sodium Carbonate as a Base for Deacetylation

Step-a) (1S)-1,5-anhydro-1-C-[4-chloro-3-[(4-ethoxyphenyl)methyl]phenyl]-D-glucitol Glycerol Solvate

A mixture of (2R,3R,4R,5S,6S)-2-(acetoxymethyl)-6-(4-chloro-3-(4-ethoxybenzyl) phenyptetrahydro-2H-pyran-3,4,5-triyltriacetate compound of formula-7 (20 gms), methanol (180 ml) and water (20 ml) was stirred for 30 mins at 25-30° C. Sodium carbonate (33.06 gms) was added to the reaction mixture at 25-30° C., heated to 45-50° C. and stirred for 8 hrs at the same temperature. Cooled the reaction mixture to 25-30° C. and stirred for 15 mins at the same temperature. Filtered the reaction mixture, washed with methanol and then distilled off the solvent from the filtrate under reduced pressure. Water was added to the obtained compound and stirred for 10 mins at 25-30° C. Decanted water layer from the reaction mixture. Ethyl acetate followed by water were added to the organic layer at 25-30° C. and stirred for 15 mins. Separated both the organic and aqueous layers, carbon (1.0 gm) was added to the organic layer and heated to 45-50° C. Cooled the reaction mixture to 25-30° C. Filtered the reaction mixture through hyflo bed, washed with ethyl acetate and distilled off the solvent from the filtrate under reduced pressure to get (1S)-1,5-anhydro-1-C-[4-chloro-3-[(4-ethoxyphenyl)methyl]phenyl]-D-glucitol compound of formula-1.

Purity: 99.1% by HPLC.

### Step-b) Preparation of Glycerol Solvate of Compound of Formula-1:

Water (600 ml) was added to the obtained compound at 30-35° C. and stirred for 20 mins. Heated the reaction mixture to 80-85° C. and stirred for 1 hr 30 mins. Cooled the reaction mixture to 25-30° C. Glycerol (3.83 gms) was added to the reaction mixture at 25-30° C. and stirred for 30 mins at the same temperature. Seed the (2S,3R,4R,5S,6R)-2-(4-chloro- 3-(4-ethoxybenzyl)phenyl)-6-(hydroxymethyl) tetrahydro-2H-pyran-3,4,5-triol glycerol solvate obtained in example-5 to the reaction mixture at 25-30° C. and stirred for 2 hrs at the same temperature. Cooled the reaction mixture to 10-15° C. and stirred for 6 hrs at the same temperature. Filtered the precipitated solid, washed with water and then dried to get (1S)-1,5-anhydro-1-C-[4-chloro-3-[(4-ethoxyphenyl)methyl]phenyl]-D-glucitol glycerol solvate. The PXRD pattern of the obtained compound is represented in FIG. 1.

Step-c) Preparation of (1S)-1,5-anhydro-1-C-[4-chloro-3-[(4-ethoxyphenyl)methyl]phenyl]-D-glucitol Compound of Formula-1

Ethyl acetate (10 ml) was added to the solid obtained in step-(b) at 25-30° C. and stirred for 15 mins at 25-30° C. Water (20 ml) was added to the reaction mixture at 25-30° C. and stirred for 20 mins at 25-30° C. Separated both the organic and aqueous layers. Distilled off the solvent completely from the organic layer under reduced pressure to get title compound as a solid.

Yield: 12 gms; Purity by HPLC: 99.6%.

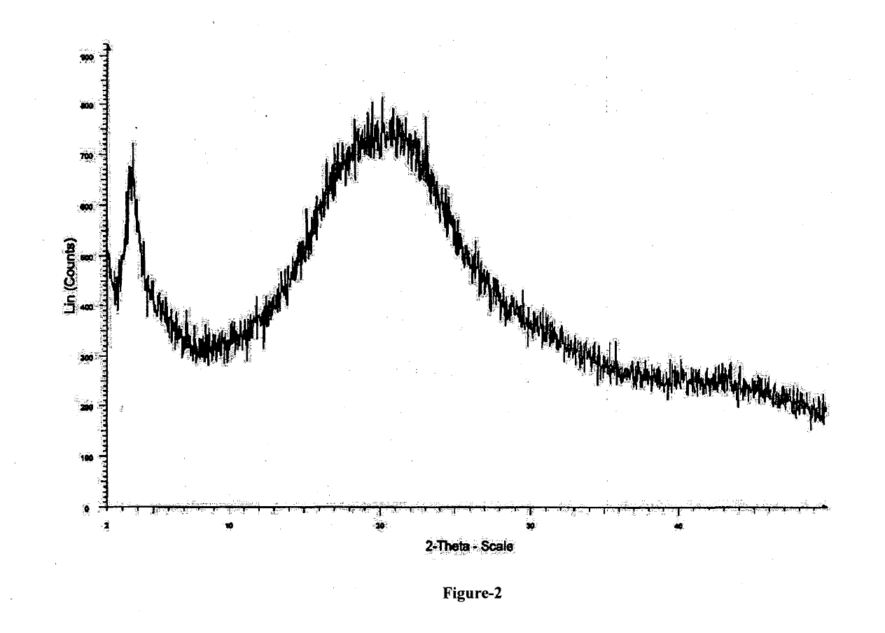

The PXRD pattern of the obtained compound is represented in FIG. 2.

### Example-7

### Preparation of (18)-1,5-anhydro-1-C-[4-chloro-3-[(4-ethoxyphenyl)methyl]phenyl]-D-glucitol Compound of Formula-1 Without Glycerol Solvate Formation Using Sodium Carbonate as a Base for Deacetylation

A mixture of (2R,3R,4R,5S,6S)-2-(acetoxymethyl)-6-(4-chloro-3 -(4-ethoxybenzyl) phenyptetrahydro-2H-pyran-3,4,5-triyl triacetate compound of formula-7 (10 gms), methanol (90 ml) and water (10 ml) was stirred for 30 mins at 25-30° C. Sodium carbonate (16.53 gms) was added to the reaction mixture, heated to 45-50° C. and stirred for 6 hrs at the same temperature. Cooled the reaction mixture to 25-30° C. and stirred for 15 mins at the same temperature. Filtered the reaction mixture, washed with methanol and distilled off the solvent completely from the filtrate under reduced pressure. Ethyl acetate followed by water were added to the obtained compound at 25-30° C. and stirred for 15 mins at the same temperature. Separated the both organic and aqueous layers, the organic layer was washed with 2% aqueous sodium bicarbonate solution, followed by 10% aqueous sodium chloride solution. Distilled off the solvent completely from the organic layer under reduced pressure to get (1S)-1,5-anhydro-1-C-[4-chloro-3-[(4-ethoxyphenyl)methyl]phenyl]-D-glucitol compound of formula-1.

Yield: 6.8 gms; % yield: 96.18%; Purity by HPLC: 99.08%.

The PXRD pattern of the obtained compound is represented in FIG. 2.

### Example-8

### Preparation of (1S)-1,5-anhydro-1-C-[4-chloro-3-[(4-ethoxyphenyl)methyl]phenyl]-D-glucitol Compound of Formula-1 Without Glycerol Solvate Using Sodium Hydroxide as a Base for Deacetylation

The (1S)-1,5-anhydro-1-C-[4-chloro-3-[(4-ethoxyphenyl)methyl]phenyl]-D-glucitol compound of formula-1 can be prepared according to example-7 starting from 5 gms of (2R,3R,4R,5S,6S)-2-(acetoxymethyl)-6-(4-chloro-3-(4-ethoxybenzyl)phenyptetrahydro-2H-pyran-3,4,5-triyl triacetate compound of formula-7 using sodium hydroxide in place of sodium carbonate. Yield: 2.6 gms; % yield: 73.68%; Purity by HPLC: 94.54%. The PXRD pattern of the obtained compound is represented in FIG. 2.

### Example-9

### Preparation of (1S)-1,5-anhydro-1-C-[4-chloro-3-[(4-ethoxyphenyl) methyl]phenyl]-D-glucitol Compound of Formula-1 Through Glycerol Solvate Formation Using Sodium Carbonate as a Base for Deacetylation

A mixture of (2R,3R,4R,5S,6S)-2-(acetoxymethyl)-6-(4-chloro-3- (ethoxybenzyl) phenyltetrahydro-2H-pyran-3,4,5-triyl triacetate compound of formula-7 (100 gm), methanol (900 ml) and water (100 ml) was stirred for 30 minutes at 25-30° C. Sodium carbonate (165.3 gm) was added to the reaction mixture at 25-30° C., heated to 45-50° C. and stirred the reaction mixture for 10 hrs at the same temperature. Cooled the reaction mixture to 25-30° C. and stirred for 15 minutes at the same temperature. Filtered the reaction mixture, washed with methanol and then distilled off the solvent completely from the filtrate under reduced pressure. Water was added to the obtained compound at 25-30° C. and stirred for 20 minutes at the same temperature. Decanted water layer from the reaction mixture. Water (1000 ml) was added to the obtained compound at 25-30° C. and stirred for 20 minutes at the same temperature. Heated the reaction mixture to 80-85° C. and stirred for 1 hr 30 minutes. Cooled the reaction mixture to 25-30° C. and stirred for 20 minutes at the same temperature. Glycerol (19.15 gm) was added to the reaction mixture at 25-30° C. and stirred the reaction mixture for 30 minutes at the same temperature. Cooled the reaction mixture to 0-5° C. and stirred for 6 hrs at the same temperature. Filtered the precipitated solid and washed with water to get (1S)-1,5-anhydro-1-C-[4-chloro-3-[(4-ethoxyphenyl)methyl]phenyl]-D-glucitol glycerol solvate. Water (3000 ml) and MTBE (1200 ml) was added to the obtained compound. Heated the reaction mixture to 45-50° C. and stirred for 20 minutes at the same temperature. Separated both the organic and aqueous layers. The organic layer was washed with 5% aq. hydrochloride solution. Distilled off the solvent from the organic layer under reduced pressure. Methanol (100 ml) and carbon (10 gm) was added to the obtained compound at 50-55° C. and stirred for 30 minutes at the same temperature. Filtered the reaction mixture through high flow and washed with methanol. Distilled off the solvent completely from the filtrate under reduced pressure and dried to get the title compound.

Yield: 61.0 gms; Purity by HPLC: 99.51%.

The PXRD pattern of the obtained compound is represented in FIG. 2.

### Example-10

### Preparation of (1S)-1,5-anhydro-1-C-[4-chloro-3-[(4-ethoxyphenyl) methyl]phenyl]-D-glucitol Compound with (S)-propane-1,2-diol (1:1) Hydrate

A mixture of (1S)-1,5-anhydro-1-C-[4-chloro-3-[(4-ethoxyphenyl)methyl]phenyl]-D-glucitol (60 gm) and isopropyl acetate (300 ml) was heated to 50-55° C. and stirred for 20 minutes at the same temperature. Cooled the reaction mixture to 25-30° C. Propane glycol (12.5 gm) and water (5 ml) was added to the reaction mixture at 25-30° C. and stirred the reaction mixture for 20 minutes at the same temperature. Cyclohexane (300 ml) was added slowly to the reaction mixture at 25-30° C. The reaction mixture was seeded with (1S)-1,5-anhydro-1-C-[4-chloro-3-[(4-ethoxyphenyl)methyl]phenyl]-D-glucitol (40 mg) and stirred the reaction mixture for 2 hrs at 25-30° . Cooled the reaction mixture to 15-20° C. and stirred the reaction mixture for 6 hrs at the same temperature. Filtered the precipitated solid and washed with the mixture of isopropyl acetate and cyclohexane. Isopropyl acetate (300 ml) was added to the obtained compound at 25-30° C. Heated the reaction mixture to 45-50° C. and stirred for 30 minutes at the same temperature. Cooled the reaction mixture to 25-30° C. and stirred the reaction mixture for 15 minutes at the same temperature. Cyclohexane (300 ml) was slowly added to the reaction mixture at 25-30° C. and stirred the reaction mixture for 2 hrs at the same temperature. Cooled the reaction mixture to 15-20° C. and stirred the reaction mixture for 2 hrs at the same temperature. Filtered the precipitated solid, washed with the mixture of isopropyl acetate and cyclohexane and dried to get the title compound. Yield: 55.0 gms; MR: 74-78° C.

Particle size distribution: D (0.1): 34.07 μm; D(0.5): 115.75 μm; D(0.9): 280.81 μm; D[4,3]: 139.10.

1-17. (canceled)

18. A process for the preparation of pure amorphous (1S)-1,5-anhydro-1-C-[4-chloro-3-[(4-ethoxy phenyl)methyl]phenyl]-D-glucitol compound of formula-1, comprising of:
a) Treating (2R,3R,4R,5S,6S)-2-(acetoxymethyl)-6-(4-chloro-3-(4-ethoxybenzyl)phenyl) tetrahydro-2H-pyran-3,4,5-triyl triacetate compound of formula-7 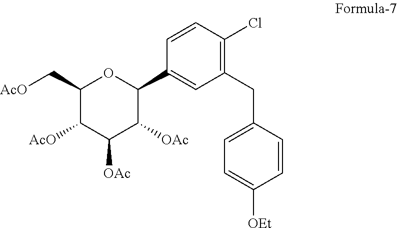 [SMILES: CCOc1ccc(Cc2cc([C@@H]3O[C@H](COC(C)=O)[C@@H](OC(C)=O)[C@H](OC(C)=O)[C@H]3OC(C)=O)ccc2Cl)cc1]
with a mild base selected from alkali metal carbonates and bicarbonates in a suitable solvent selected from ether solvents, ester solvents, alcoholic solvents, chloro solvents, polar aprotic solvents, ketone solvents, hydrocarbon solvents, polar solvents, nitrile solvents or mixtures thereof to provide (1S)-1,5-anhydro-1-C-[4-chloro-3-[(4-ethoxyphenyl)methyl]phenyl]-D-glucitol compound of formula-1, 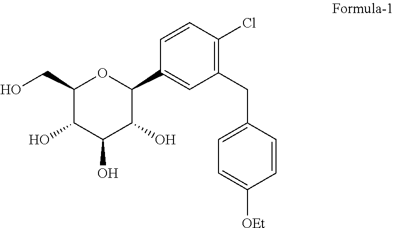 [SMILES: CCOc1ccc(Cc2cc([C@@H]3O[C@H](CO)[C@@H](O)[C@H](O)[C@H]3O)ccc2Cl)cc1]
b) converting the compound of formula-1 into its glycerol solvate by treating it with glycerol in a suitable solvent selected from ether solvents, ester solvents, chloro solvents, polar aprotic solvents, ketone solvents, hydrocarbon solvents, polar solvents, nitrile solvents, alcoholic solvents or mixtures thereof to provide its glycerol solvate, 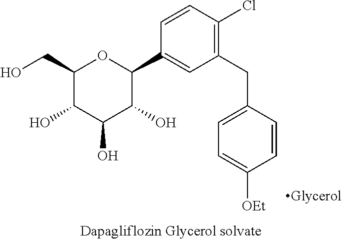 [SMILES: CCOc1ccc(Cc2cc([C@@H]3O[C@H](CO)[C@@H](O)[C@H](O)[C@H]3O)ccc2Cl)cc1]
c) dissolving the glycerol solvate in a suitable solvent selected from ether solvents, ester solvents, chloro solvents, polar aprotic solvents, ketone solvents, hydrocarbon solvents, nitrile solvents or mixtures thereof, washing the reaction mixture with water and then distilling off the solvent to provide pure amorphous compound of formula-1.

19. The process according to claim-1, wherein the process for the preparation of pure amorphous (1S)-1,5-anhydro-1-C-[4-chloro-3-[(4-ethoxy phenyl)methyl]phenyl]-D-glucitol compound of formula-1, comprising of:
a) Treating (2R,3R,4R,5S,6S)-2-(acetoxymethyl)-6-(4-chloro-3-(4-ethoxybenzyl)phenyl) tetrahydro-2H-pyran-3,4,5-triyl triacetate compound of formula-7 with sodium carbonate in aqueous methanol to provide (1S)-1,5-anhydro-1-C-[4-chloro-3-[(4-ethoxyphenyl) methyl]phenyl]-D-glucitol compound of formula-1,
b) converting the compound of formula-1 into its glycerol solvate by treating it with glycerol in water,
c) dissolving the glycerol solvate in methyl tertiarybutyl ether (MTBE), washing the reaction mixture with water and then distilling off the solvent to provide pure amorphous compound of formula-1.

20. The process according to claim-1, wherein the process for the preparation of (1S)-1,5-anhydro-1-C-[4-chloro-3-[(4-ethoxyphenyl)methyl]phenyl]-D-glucitol compound of formula-1, comprising of treating (2R,3R, 4R,5S,6S)-2-(acetoxymethyl)-6-(4-chloro-3-(4-ethoxybenzyl)phenyptetrahydro-2H-pyran-3,4,5-triyl triacetate compound of formula-7 using a mild base selected from alkali metal carbonates and bicarbonates in a suitable solvent selected from hydrocarbon solvents, ether solvents, ester solvents, polar aprotic solvents, alcoholic solvents, ketone solvents, chloro solvents, polar solvents, nitrile solvents or mixtures thereof to provide compound of formula-1.

21. The process according to claim-3, wherein the process for the preparation of (1S)-1,5-anhydro-1-C-[4-chloro-3-[(4-ethoxyphenyl)methyl]phenyl]-D-glucitol compound of formula-1, comprising of treating (2R,3 R, 4R,5S,6S)-2-(acetoxymethyl)-6-(4-chloro-3-(4-ethoxybenzyl)phenyptetrahydro-2H-pyran-3,4,5-triyl triacetate compound of formula-7 with sodium carbonate in aqueous methanol to provide compound of formula-1.

22. Glycerol solvate of (1S)-1,5-anhydro-1-C-[4-chloro-3-[(4-ethoxyphenyl)methyl]phenyl]-D-glucitol.

23. The glycerol solvate of (1S)-1,5-anhydro-1-C-[4-chloro-3-[(4-ethoxyphenyl)methyl]phenyl]-D-glucitol as claimed in claim-5 is a crystalline solid.

24. The Crystalline form-M of (1S)-1,5-anhydro-1-C-[4-chloro-3-[(4-ethoxyphenyl)methyl]phenyl]-D-glucitol glycerol solvate as claimed in claim-6 is characterized by its powder X-ray diffraction pattern having peaks at about 4.1, 16.2, 20.3, 20.6 and 24.8±0.2 degrees of 2-theta.

25. The crystalline form-M of (1S)-1,5-anhydro-1-C-[4-chloro-3-[(4-ethoxyphenyl)methyl]phenyl]-D-glucitol glycerol solvate as claimed in claim-7, is prepared by a process comprising of treating the (1S)-1,5-anhydro-1-C-[4-chloro-3-[(4-ethoxyphenyl) methyl]phenyl]-D-glucitol compound of formula-1 with glycerol in a suitable solvent selected from ether solvents, ester solvents, polar aprotic solvent, alcoholic solvents, polar aprotic solvents, chloro solvents, ketone solvents, polar solvent, nitrile solvents or mixtures thereof.

26. The process according to claim-8, wherein the process for the preparation of crystalline form-M of (1S)-1,5-anhydro-1-C-[4-chloro-3-[(4-ethoxyphenyl)methyl]phenyl]-D-glucitol glycerol solvate, comprising of treating the (1S)-1,5-anhydro-1-C-[4-chloro-3- [(4-ethoxyphenyl)methyl]phenyl]-D-glucitol compound of formula-1 with glycerol in water.

27. A process for the preparation of pure (2S,3R,4R,5S,6R)-2-(4-chloro-3-(4-ethoxybenzyl)phenyl)-6-(hydroxymethyl)tetrahydro-2H-pyran-3,4,5-triol compound of formula-1, comprising of:
a) Reacting the (3R,4S,5S,6R)-3,4,5-trihydroxy-6-(hydroxymethyl)tetrahydro-2H-pyran-2-one compound of formula-8 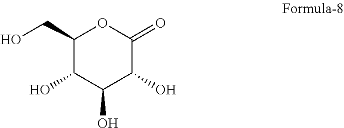 [SMILES: O=C1O[C@H](CO)[C@@H](O)[C@H](O)[C@H]1O]
with trimethyl silyl chloride in presence of N-methyl morpholine in tetrahydrofuran to provide (3R,4S,5R,6R)-3,4,5-tris(trimethylsilyloxy)-6-((trimethylsilyloxy)methyl) tetrahydro-2H-pyran-2-one compound of formula-5, 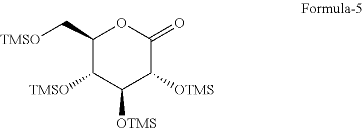
b) reacting the compound of formula-5 in-situ with 4-bromo-1-chloro-2-(4-ethoxybenzyl) benzene compound of formula-4 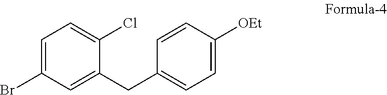 [SMILES: CCOc1ccc(Cc2cc(Br)ccc2Cl)cc1]
in presence of n-butyl lithium in tetrahydrofuran, followed by treating the obtained compound with methane sulfonic acid in methanol to provide (2S,3R,4S,5S,6R)-2-(4-chloro-3-(4-ethoxybenzyl)phenyl)-6-(hydroxymethyl)-2-methoxy tetrahydro-2H-pyran-3,4,5- triol compound of formula-6, 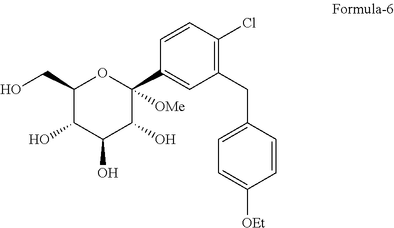 [SMILES: CCOc1ccc(Cc2cc([C@]3(OC)O[C@H](CO)[C@@H](O)[C@H](O)[C@H]3O)ccc2Cl)cc1]
c) reacting the compound of formula-6 with triethylsilane in presence of BF₃-etherate in a mixture of dichloromethane and acetonitrile to provide (1S)-1,5-anhydro-1-C-[4-chloro-3-[(4-ethoxyphenyl)methyl]phenyl]-D-glucitol compound of formula-1,
d) reacting the compound of formula-1 in-situ with acetic anhydride in presence of dimethylamino pyridine in dichloromethane, purifying the obtained compound using methanol to provide (2R,3R,4R,5S,6S)-2-(acetoxymethyl)-6-(4-chloro-3-(4-ethoxy benzyl)phenyl)tetrahydro-2H-pyran-3,4,5-triyl triacetate compound of formula-7, 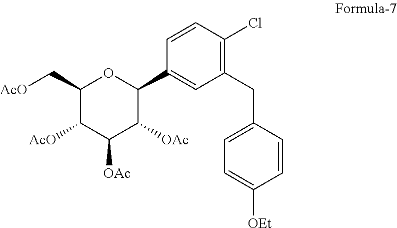 [SMILES: CCOc1ccc(Cc2cc([C@@H]3O[C@H](COC(C)=O)[C@@H](OC(C)=O)[C@H](OC(C)=O)[C@H]3OC(C)=O)ccc2Cl)cc1]
e) treating the compound of formula-7 with a mild base selected from alkali metal carbonates and bicarbonates in a suitable solvent selected from ether solvents, ester solvents, chloro solvents, polar aprotic solvents, ketone solvents, hydrocarbon solvents, nitrile solvents or mixtures thereof to provide compound of formula-1,
f) converting the compound of formula-1 into its glycerol solvate by treating it with glycerol in a suitable solvent selected from ether solvents, ester solvents, chloro solvents, polar aprotic solvents, ketone solvents, hydrocarbon solvents, alcoholic solvents, polar solvents, nitrile solvents or mixtures thereof,
g) dissolving the glycerol solvate in a suitable solvent selected from ether solvents, ester solvents, chloro solvents, polar aprotic solvents, ketone solvents, hydrocarbon solvents, nitrile solvents or mixtures thereof, washing the reaction mixture with water and then distilling off the solvent to provide pure compound of formula-1.

28. The process according to claim 10, wherein, the 4-bromo-1-chloro-2-(4-ethoxy benzyl)benzene compound of formula-4 can be prepared by the following steps of:
a) Converting the 5-bromo-2-chlorobenzoic acid compound of formula-2 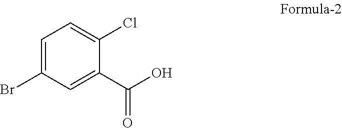 [SMILES: O=C(O)c1cc(Br)ccc1Cl]
into its acid chloride by treating it with thionyl chloride in a mixture of dichloromethane and dimethylformamide,
b) reacting the acid chloride in-situ with phenetole in presence of aluminium chloride in dichloromethane, purifying the obtained compound using methanol to provide (5-bromo-2-chlorophenyl)(4-ethoxyphenyl)methanone compound of formula-3, 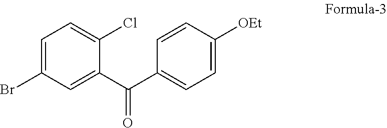 [SMILES: CCOc1ccc(C(=O)c2cc(Br)ccc2Cl)cc1]
c) reducing the compound of formula-3 with triethylsilane in presence of titanium tetrachloride in dichloromethane, purifying the obtained compound using methanol to provide 4-bromo-1-chloro-2-(4-ethoxybenzyl)benzene compound of formula-4.

29. The process according to claim-10, wherein the process for the preparation of pure amorphous (1S)-1,5-anhydro-1-C-[4-chloro-3-[(4-ethoxyphenyl)methyl]phenyl]-D-glucitol compound of formula-1, comprising of:
a) Reacting the (3R,4S,5S,6R)-3,4,5-trihydroxy-6-(hydroxymethyl)tetrahydro-2H-pyran-2-one compound of formula-8 with trimethyl silyl chloride in presence of N-methylmorpholine in tetrahydrofuran to provide (3R,4S,5R,6R)-3,4,5-tris(trimethyl silyloxy)-6-((trimethylsilyloxy)methyl)tetrahydro-2H-pyran-2-one compound of formula-5,
b) reacting the compound of formula-5 in-situ with 4-bromo-1-chloro-2-(4-ethoxybenzyl) benzene compound of formula-4 in presence of n-butyl lithium in tetrahydrofuran, followed by treating the obtained compound with methane sulfonic acid in methanol to provide (2S,3R,4S,5S,6R)-2-(4-chloro-3-(4-ethoxybenzyl)phenyl)-6- (hydroxymethyl)-2-methoxy tetrahydro-2H-pyran-3,4,5-triol compound of formula-6,
c) reacting the compound of formula-6 with triethyl silane in presence of BF₃-etherate in a mixture of dichloromethane and acetonitrile to provide (1S)-1,5-anhydro-1-C-[4-chloro-3-[(4-ethoxyphenyl)methyl]phenyl]-D-glucitol compound of formula-1,
d) reacting the compound of formula-1 in-situ with acetic anhydride in presence of dimethylamino pyridine in dichloromethane, purifying the obtained compound using methanol to provide (2R,3R,4R,5S,6S)-2-(acetoxymethyl)-6-(4-chloro-3-(4-ethoxy benzyl)phenyl)tetrahydro-2H-pyran-3,4,5-triyl triacetate compound of formula-7,
e) treating the compound of formula-7 with sodium carbonate in aqueous methanol to provide compound of formula-1,
f) converting the compound of formula-1 into its glycerol solvate by treating it with glycerol in water,
g) dissolving the glycerol solvate in ethyl acetate, washing the reaction mixture with water and then distilling off the solvent to provide pure amorphous compound of formula-1.

30. The process according to claim 10, wherein, the process for the preparation of 4-bromo-1-chloro-2-(4-ethoxy benzyl)benzene compound of formula-4 comprising of, reducing the (5-bromo-2-chlorophenyl)(4-ethoxyphenyl)methanone compound of formula-3 with triethylsilane in presence of titanium tetrachloride in dichloromethane, optionally purifying the obtained compound using methanol to provide 4-bromo-1-chloro-2-(4-ethoxybenzyl)benzene compound of formula-4.

31. The (1S)-1,5-anhydro-1-C-[4-chloro-3-[(4-ethoxyphenyl)methyl]phenyl]-D-glucitol compound of formula-1 obtained according to claim-1 having purity more than 99% by HPLC, preferably more than 99.6% purity by HPLC.

32. Use of glycerol solvate of (1S)-1,5-anhydro-1-C-[4-chloro-3 -[(4-ethoxyphenyl) methyl]phenyl]-D-glucitol compound of formula-1 as claimed in claim-5 of the present invention can be utilized for the preparation of amorphous (1S)-1,5-anhydro-1-C-[4-chloro-3-[(4-ethoxyphenyl)methyl]phenyl]-D-glucitol compound of formula-1.

33. Use of pure (1S)-1,5-anhydro-1-C-[4-chloro-3-[(4-ethoxyphenyl)methyl]phenyl]-D-glucitol compound of formula-1 and its glycerol solvate crystalline form-M as claimed in claim-7 of the present invention can be utilized for the preparation of (1S)-1,5-anhydro-1-C-[4-chloro-3-[(4-ethoxyphenyl)methyl]phenyl]-D-glucitol(S)-1,2-propane-diol monohydrate.

34. Use of pure (1S)-1,5-anhydro-1-C-[4-chloro-3-[(4-ethoxyphenyl)methyl]phenyl]-D-glucitol compound of formula-1 and its glycerol solvate crystalline form-M of the present invention can be utilized for the preparation of pharmaceutical composition.
